(19) United States
(12) Patent Application Publication
(10) Pub. No.: US 2019/0374188 A1
(43) Pub. Date: Dec. 12, 2019

(54) BIOLOGICAL DATA PROCESSING DEVICE, BIOLOGICAL DATA PROCESSING SYSTEM AND BIOLOGICAL DATA PROCESSING PROGRAM

(71) Applicant: Hideaki YAMAGATA, Kanagawa (JP)

(72) Inventor: Hideaki YAMAGATA, Kanagawa (JP)

(73) Assignee: Ricoh Company, Ltd., Tokyo (JP)

(21) Appl. No.: 16/463,566

(22) PCT Filed: Nov. 28, 2017
(86) PCT No.: PCT/JP2017/042633

§ 371 Date: May 23, 2019

(30) Foreign Application Priority Data
Dec. 2, 2016 (JP) 2016-235112

(51) Int. Cl. A61B 6/00; A61B 5/04; A61B 5/00; G16H 30/20; G16H 40/63

(52) U.S. Cl. CPC A61B 6/5217; A61B 6/463; A61B 6/505; A61B 5/04005; A61B 2090/3954; A61B 5/0035; A61B 6/5247; G16H 30/20; G16H 40/63; A61B 5/407

(57) ABSTRACT

A disclosed biological data processing device includes a calculator configured to calculate relative position data indicating a relative position of a subject with respect to a biological sensor for measuring the subject using the biological sensor, a specifying unit configured to identify a predetermined part of the subject to specify a relative position of the identified predetermined part with respect to the biological sensor based on the relative position data, and a generator configured to reconfigure electric current sources based on biological data measured by the biological sensor, using the relative position specified by the specifying unit as a calculated position, to generate reconfigured data.

### TECHNICAL FIELD

The disclosure discussed herein relate to a biological data processing device, a biological data processing system and a biological data processing program.

### BACKGROUND ART

The related art technology suggests a biological data processing device that visualizes neural activity of a subject based on biological data measured with a biological sensor. An example of such biological data processing device may include a magnetic field data processing device configured to measure current flowing through nerves in a spine of a subject as magnetic field data with a magnetic sensor and reconfigure electric current sources on a mesh-unit basis so as to generate reconfigured data. According to the magnetic field data processing device, neural activity in the spine of the subject may be visualized as reconfigured data, which may assist a physician or the like to specify a damaged part in the spine of the subject.

### CITATION LIST

### Patent Literature

PTL 1: Japanese Unexamined Patent Application Publication No. H5-197767

### SUMMARY OF INVENTION

### Technical Problem

In such a magnetic field data processing device, reduction in the number of the grid points by an increase in a mesh size for generating the reconfigured data will lower the accuracy of the reconfigured data. In contrast, an increase in the number of grid points by a decrease in a mesh size will increase the computational time required for reconfiguration and lower resistance to artifacts. Accordingly, an appropriate number of grid points may be required for generating the reconfigured data.

### Solution to Problem

The present invention has been made in view of the above-described complications; it is an object of the present invention to generate reconfigured data suitable for identifying a damaged part of a subject.

According to an aspect of an embodiment, a biological data processing device includes the following configuration; that is, the biological data processing device includes a calculator configured to calculate relative position data indicating a relative position of a subject with respect to a biological sensor for measuring the subject using the biological sensor; a specifying unit configured to identify a predetermined part of the subject to specify a relative position of the identified predetermined part with respect to the biological sensor based on the relative position data; and a generator configured to reconfigure electric current sources based on biological data measured by the biological sensor, using the relative position specified by the specifying unit as a calculated position, to generate reconfigured data.

### Advantageous Effect of the Invention

According to the embodiment of the present invention, it is possible to generate reconfigured data suitable for specifying a damaged part of a subject.

### BRIEF DESCRIPTION OF DRAWINGS

FIG. 1 is a diagram illustrating an example of an overall configuration of a magnetic field data processing system;

FIG. 2 is a diagram illustrating an external configuration of an X-ray imaging unit and an example of X-ray image data;

FIG. 3 is a diagram illustrating an example of an external configuration of a magnetic sensor array and magnetic field data;

FIG. 4 is a diagram schematically illustrating a current flowing through a nerve in a spine of a subject;

FIG. 5 is a diagram illustrating an example of a hardware configuration of a magnetic field data processing device;

FIG. 6 is a flowchart illustrating a flow of a subject measurement process by a magnetic field data processing system;

FIG. 7 is a flowchart illustrating a flow of a relative position calculation process by a magnetic field data processing system;

FIG. 8 is a flowchart illustrating a flow of a coordinate-added X-ray image data calculation process by a magnetic field data processing device;

FIG. 9 is a diagram schematically illustrating a flow of a relative position calculation process (including a coordinate-added X-ray image data calculation process by a magnetic field data processing device) by a magnetic field data processing system;

FIG. 10 is a diagram illustrating a detailed functional configuration of a mesh generator configured to execute a mesh generation process;

FIG. 11 is a flowchart illustrating a flow of a mesh generation process by respective units of a mesh generator;

FIG. 12A is a diagram schematically illustrating a flow of a mesh generation process by respective units of a mesh generator;

FIG. 12B is a diagram schematically illustrating a flow of a mesh generation process by respective units of the mesh generator;

FIG. 12C is a diagram schematically illustrating a flow of a mesh generation process by respective units of the mesh generator;

FIG. 12D is a diagram schematically illustrating a flow of a mesh generation process by respective units of a mesh generator;

FIG. 13 is a flowchart illustrating a flow of a reconfiguration process by a magnetic field data processing system;

FIG. 14 is a flowchart illustrating a flow of a reconfigured data generation process by a magnetic field data processing device; and

FIG. 15 is a diagram schematically illustrating a flow of a reconfiguration process (including a reconfigured data generation process by a magnetic field data processing device) by a magnetic field data processing system.

### DESCRIPTION OF EMBODIMENTS

The following illustrates detailed description of embodiments of the present invention with reference to the accompanying drawings. In the specification and the drawings according to the embodiments, the same reference numerals are assigned to constituent elements having substantially the same functional configurations, and duplicated explanation will be omitted.

### Embodiments

### 1. Overall Configuration of Magnetic Field Data Processing System

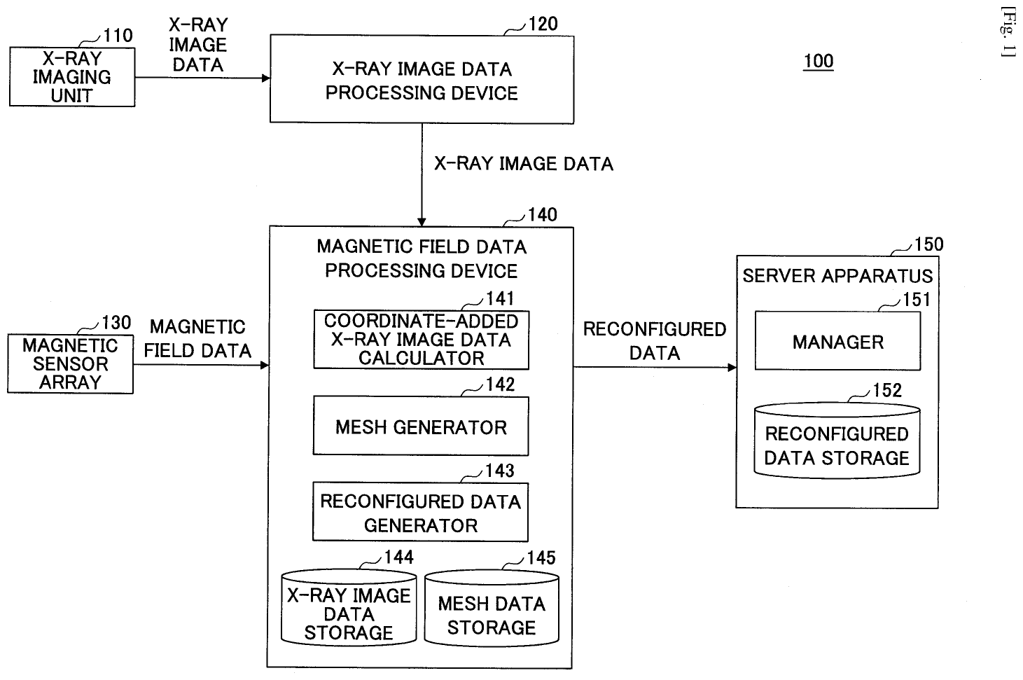

First, a description is given of an overall configuration of a magnetic field data processing system which is an example of a biological data processing system. FIG. 1 is a diagram illustrating an example of an overall configuration of a magnetic field data processing system.

As illustrated in FIG. 1, the magnetic field data processing system 100 includes an X-ray imaging unit 110, an X-ray image data processing device 120, a magnetic sensor array 130, a magnetic field data processing device 140, and a server apparatus 150.

The X-ray imaging unit 110 irradiates a subject with X-rays from the front of the subject with position detection markers (e.g., marker coils) attached to the subject and detects X-rays transmitted through the subject (i.e., performing X-ray radiography) to generate X-ray image data. The X-ray imaging unit 110 transmits the generated X-ray image data to the X-ray image data processing device 120.

The X-ray image data processing device 120 performs various image processing such as noise removal on the X-ray image data received from the X-ray imaging unit 110, and transmits the processed X-ray image data to the magnetic field data processing device 140.

The magnetic sensor array 130 is a biological sensor having multiple magnetic sensors arranged in an array, each of which measures two types of magnetic field data in the present embodiment. First, the magnetic sensor array 130 according to the present embodiment measures magnetic field data used for generating coordinate-added X-ray image data (details will be described later). Specifically, the magnetic sensor array 130 measures the magnetic field data in a state where marker coils are attached to the subject. Secondly, the magnetic sensor array 130 according to the present embodiment delivers predetermined electrical stimuli to the subject with the marker coils being removed, and measures a current flowing through the nerve in the spine of the subject as magnetic field data.

Note that the magnetic field data measured in each of the magnetic sensors included in the magnetic sensor array 130 are input to the magnetic field data processing device 140.

The magnetic field data processing device 140 is an example of a biological data processing device, and a magnetic field data processing program, which is an example of a biological data processing program, is installed in the magnetic field data processing device 140. Executing the magnetic field data processing program causes the magnetic field data processing device 140 to function as the coordinate-added X-ray image data calculator 141, the mesh generator 142, and the reconfigured data generator 143.

The coordinate-added X-ray image data calculator 141 is an example of a calculator. The coordinate-added X-ray image data calculator 141 receives X-ray image data transmitted by the X-ray image data processing device 120. Further, the coordinate-added X-ray image data calculator 141 generates magnetic field distribution data based on the magnetic field data measured by the magnetic sensor array 130 with the marker coils being attached to the subject. Furthermore, based on the generated magnetic field distribution data, the coordinate-added X-ray image data calculator 141 adds coordinates indicating a relative positional relationship with respect to the magnetic sensor array 130 to the X-ray image data, thereby generating “coordinate-added X-ray image data” to store the generated coordinate-added X-ray image data in the X-ray image data storage 144.

The mesh generator 142 is an example of a specifying unit. The mesh generator 142 analyzes the coordinate-added X-ray image data stored in the X-ray image data storage 144 to identify a predetermined part of the subject (the part that a physician or the like desires to observe in order to identify a damaged part), and generates a mesh based on the identified part. The mesh generator 142 specifies a position of each of grid points of the generated mesh based on the coordinate-added X-ray image data to specify the mesh data, and stores the specified mesh data in the mesh data storage 145.

The reconfigured data generator 143 is an example of a generator. The reconfigured data generator 143 processes the magnetic field data measured by the magnetic sensor array 130 by delivering predetermined electric stimuli to the subject with the marker coils being removed, and generates reconfigured data indicating change in the current flowing through the spine of the subject. The reconfigured data generator 143 uses the mesh data stored in the mesh data storage 145 for generating the reconfigured data. In addition, the reconfigured data generator 143 transmits the reconfigured data generated by using the mesh data to the server apparatus 150.

As described above, the magnetic field data processing device 140 uses a mesh generated based on a predetermined part of the subject for generating the reconfigured data, and hence, the magnetic field data processing device 140 sets a part that the physician or the like desires to observe for identifying the damaged part as a calculated position for generating the reconfigured data. That is, the magnetic field data processing device 140 according to the present embodiment may reconfigure the electric current sources at the calculated position suitable for identifying the damaged part of the subject to generate the reconfigured data.

The server apparatus 150 is an information processing apparatus configured to manage various data. A management program is installed in the server apparatus 150, and executing the management program causes the server apparatus 150 to function as the manager 151.

The manager 151 receives reconfigured data transmitted by the magnetic field data processing device 140 and stores the received reconfigured data in the reconfigured data storage 152. Note that the server apparatus 150 may be connected to a network, for example. Further, the manager 151 is configured to transmit, upon reception of a transmission request for reconfigured data of a specific subject via a network, the requested reconfigured data of the subject to a request source.

In the example of FIG. 1, the X-ray image data processing device 120 and the magnetic field data processing device 140 are depicted as separate bodies; however, the X-ray image data processing device 120 and the magnetic field data processing device 140 may be configured to be integrated. Alternatively, part of the functions of the magnetic field data processing device 140 may be included in the X-ray image data processing device 120.

In the example of FIG. 1, the X-ray image data processing device 150 and the magnetic field data processing device 140 are depicted as separate bodies; however, the X-ray image data processing device 150 and the magnetic field data processing device 140 may be configured to be integrated.

### 2. External Configuration of X-Ray Imaging Unit and X-Ray Image Data

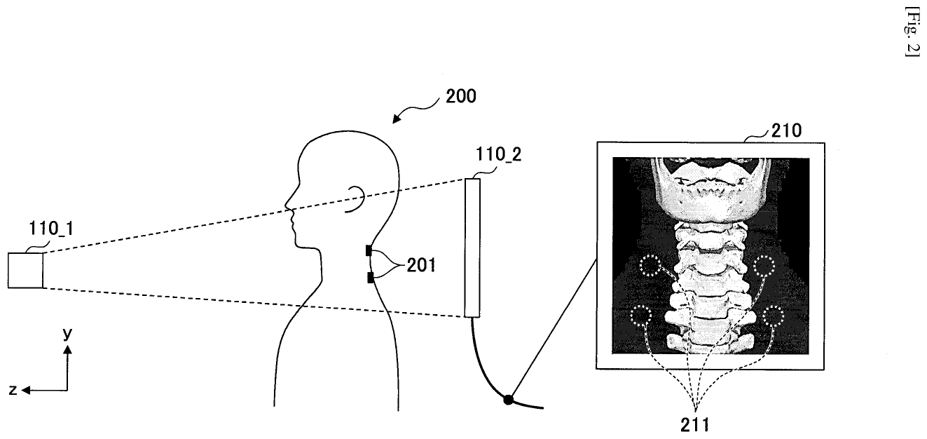

Next, an external configuration of the X-ray imaging unit 110 and the X-ray image data will be described. FIG. 2 is a diagram illustrating an external configuration of an X-ray imaging unit and an example of X-ray image data. In the present embodiment, xyz axes may be defined as follows.
A y axis is defined as an axis from the chest to the head of a subject 200 to be measured.
A z axis is defined as an axis from the back to the chest of the subject 200 to be measured.
An x axis is defined as an axis from the right arm to the left arm of the subject 200 to be measured.

As illustrated in FIG. 2, the X-ray imaging unit 110 has an X-ray source 110_1 and an X-ray detector 110_2, and is configured to perform X-ray photography by irradiating the subject 200 with X-rays from the front of the subject 200 and output X-ray image data 210.

As described above, the marker coils 201 are attached to the subject 200 for performing X-ray photography by the X-ray imaging unit 110. Hence, marker coils appear in the X-ray image data 210 (see reference numeral 211).

### 3. External Configuration of Magnetic Sensor Array and Magnetic Field Data

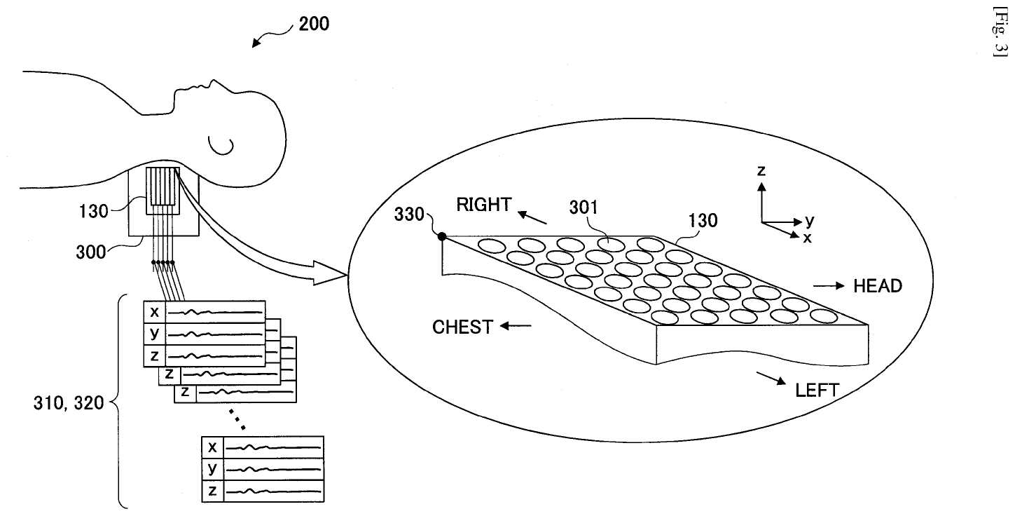

Next, an external configuration of a magnetic sensor array 130 and magnetic field data will be described. FIG. 3 is a diagram illustrating an example of an external configuration of a magnetic sensor array and magnetic field data.

As shown in FIG. 3, the magnetic sensor array 130 is disposed in a dewar 300. The dewar 300 is filled with liquid helium, and is cooled for operating the magnetic sensor array 130 at an extremely low temperature.

The magnetic sensor array 130 includes multiple magnetic sensors (7×5 magnetic sensors in the example of FIG. 3), and each of the magnetic sensors 301 outputs magnetic field data as a voltage signal in a corresponding one of the x axis, y axis, and z axis directions. In the present embodiment, voltage signals in the directions output by measuring the magnetic field emitted from the marker coils 201 by respective magnetic sensors 301 are referred to as magnetic field data 310. In addition, voltage signals in respective directions, which are output by delivering electrical stimuli to the subject 200 with the marker coils 201 being removed and measuring the current flowing through the nerve in the spine of the subject 200, are referred to as magnetic field data 320.

In the present embodiment, the position of a point 330 on the magnetic sensor array 130 (see FIG. 3) is described as the origin of the x, y, and z axes. Setting of the position of the point 330 on the magnetic sensor array 130 as the origin of the x, y, and z axes enable all the relative positional relationships with the magnetic sensor array 130 to be represented by x, y, and z coordinates.

### 4. Current Flowing Through Nerve in Spine of Subject

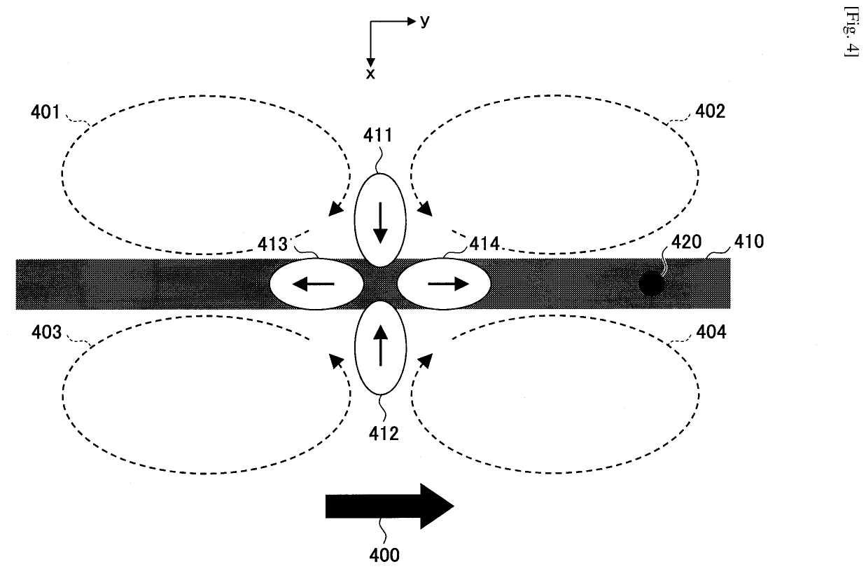

Note that a current flowing through the nerve in the spine of the subject 200 will be briefly described by delivering electrical stimuli to the subject 200. FIG. 4 is a diagram schematically illustrating a current flowing through a nerve in a spine of a subject. In FIG. 4, a bold solid line arrow 400 indicates a direction in which neural activity moves. As illustrated in FIG. 4, in a case where an electric stimulus is delivered to a predetermined part of the subject 200, neural activity of a nerve 410 in the spine of the subject 200 moves in the y-axis direction (direction toward the head of the subject 200).

Curves 401 to 404 conceptually represent current circuits in a body of the subject 200. As illustrated in FIG. 4, in the body of the subject 200, a current flows in the nerve 410 and returns after circulating around cells outside the nerve 410.

That is, the current flowing in the current circuits in the body of the subject 200 includes the current (referred to as “volume current”) flowing in the directions of the arrows 411 and 412 with respect to the nerve 410 and the current (referred to as “intracellular current”) flowing in the directions of arrows 413 and 414 within the nerve 410.

Among the above, with respect to the current flowing in the nerve 410, the intracellular current flowing in the direction of the arrow 413 is paired with the intracellular current flowing in the direction of the arrow 414. The paired intracellular currents flowing in the directions of the arrows 413 and 414 flow through the nerve 410 as a whole and are transmitted in the y-axis direction (the direction of the arrow 400).

Accordingly, when the intracellular current transmitted in the direction of the arrow 400 is observed at, for example, an observation point 420, the intracellular current flowing in the direction of the arrow 414 passes first and the intracellular current flowing in the direction of the arrow 413 passes next. As a result, at the observation point 420, an upward current is first observed, and a downward current is subsequently observed.

The magnetic sensor array 130 measures the magnetic field generated by the flow of the volume current and the intracellular current and outputs the measured magnetic field as a voltage signal. The magnetic field data processing device 140 reconfigures electric current sources (the volume current, the intracellular current) on the basis of the voltage signal outputted by the magnetic sensor array 130 and calculates a current value at predetermined observation points (each grid point included in the mesh) within the nerve 410.

### 5. Hardware Configuration of Each Device

Next, the hardware configuration of each device (the X-ray image data processing device 120, the magnetic field data processing device 140, the server apparatus 150) constituting the magnetic field data processing system 100 will be described. Since the hardware configurations of the respective devices are substantially equal, the hardware configuration of the magnetic field data processing device 140 will be described as an example for simplicity of explanation.

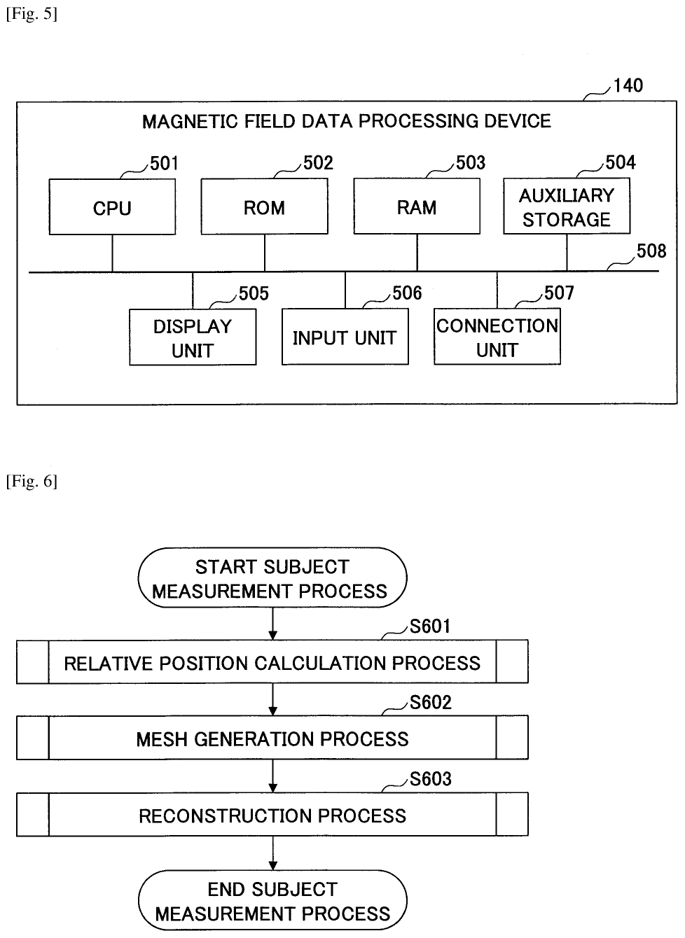

FIG. 5 is a diagram illustrating an example of a hardware configuration of a magnetic field data processing device. As illustrated in FIG. 5, the magnetic field data processing device 140 includes a CPU (Central Processing Unit) 501, a ROM (Read Only Memory) 502, and a RAM (Random Access Memory) 503. The CPU 501, the ROM 502, and the RAM 503 form what may be termed as a computer. The magnetic field data processing device 140 further includes an auxiliary storage 504, a display unit 505, an input unit 506, and a connection unit 507. Note that the respective units of the magnetic field data processing device 140 are mutually connected via a bus 508.

The CPU 501 is a device that executes various programs (e.g., a magnetic field data process program) stored in the auxiliary storage 504.

The ROM 502 is a nonvolatile main storage device. The ROM 502 stores various programs, data, and the like necessary for the CPU 501 to execute various programs stored in the auxiliary storage 504. More specifically, the ROM 302 stores a boot program such as BIOS (Basic Input/Output System) or EFI (Extensible Firmware Interface).

The RAM 503 is a volatile main storage device such as DRAM (Dynamic Random

Access Memory) or SRAM (Static Random Access Memory). The RAM 503 provides a work area, in which various programs stored in the auxiliary storage 504 are loaded upon being executed by the CPU 501.

The auxiliary storage 504 is an auxiliary storage device that stores various programs executed by the CPU 501.

The display unit 505 is a display device for displaying various screens. The input unit 506 is an input device for inputting various types of information to the magnetic field data processing device 140. The connection unit 507 is a connection device for connecting each of the magnetic sensor array 130, the X-ray image data processing device 120 and the server apparatus 150 to the magnetic field data processing device 140.

### 6. Flow of Subject Measurement Process by Magnetic Field Data Processing System

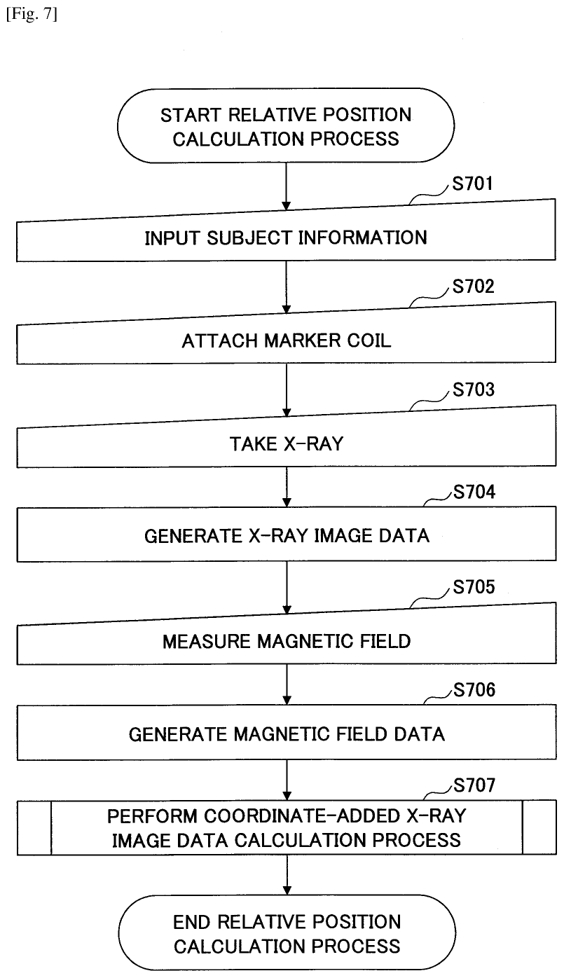

Next, a description is given of a flow of the subject measurement process for measuring the subject 200 using the magnetic field data processing system 100. FIG. 6 is a flowchart illustrating a flow of a subject measurement process by a magnetic field data processing system.

In step S601, the magnetic field data processing system 100 executes a “relative position calculation process” for calculating a relative position between the magnetic sensor array 130 and the subject 200. As a result, the magnetic field data processing system 100 generates coordinate-added X-ray image data (X-ray image data added with an x coordinate and a y coordinate having the point 330 as the origin).

In step S602, the magnetic field data processing system 100 executes a “mesh generation process” to generate a mesh used for reconfiguring electric current sources from the magnetic field data measured by the magnetic sensor array 130 based on a predetermined part of the subject. As a result, the magnetic field data processing system 100 specifies mesh data.

In step S603, the magnetic field data processing system 100 measures the subject 200 using the magnetic sensor array 130 and executes the reconfiguration process for reconfiguring the electric current sources using the mesh data. As a result, the magnetic field data processing system 100 generates reconfigured data.

Details of each of the processes (a relative position calculation process (step S601), a mesh generation process (step S602), and a reconfiguration process (step S603)) included in the subject measurement process (FIG. 6) will be described with reference to specific examples.

### 7. Illustration of Relative Position Calculation Process (Step S601)

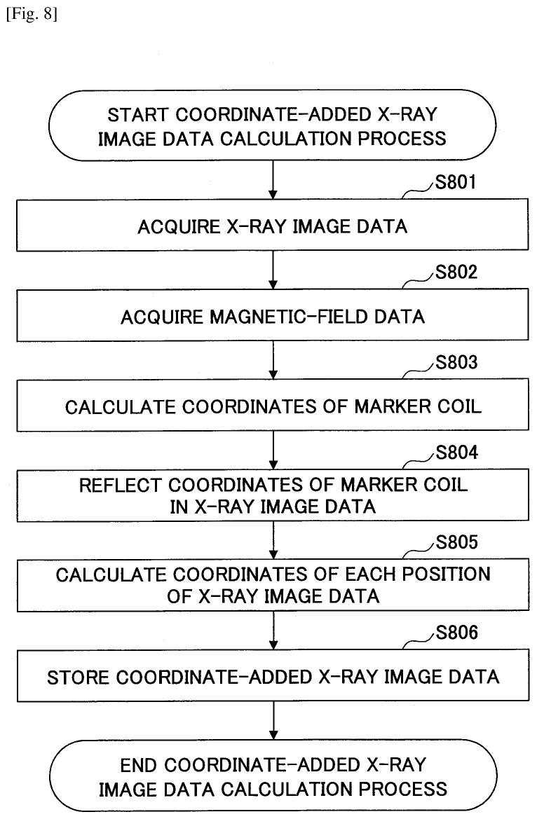

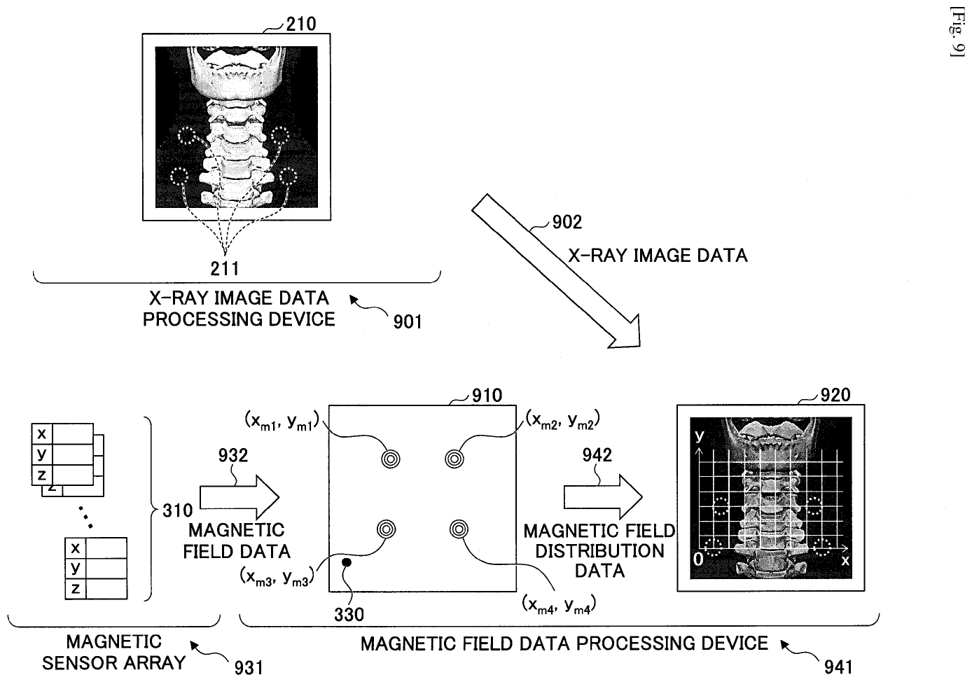

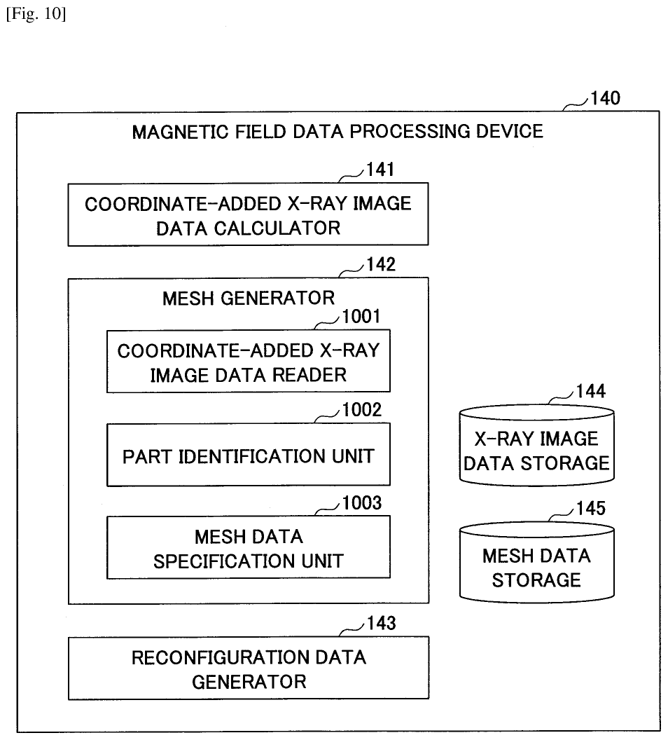

Initially, a relative position calculation process (step S601) will be described in detail using FIGS. 7 and 8, with reference to FIG. 9. FIG. 7 is a flowchart illustrating a flow of a relative position calculation process by a magnetic field data processing system. FIG. 8 is a flowchart illustrating a flow of a coordinate-added X-ray image data calculation process by a magnetic field data processing device. FIG. 9 is a diagram schematically illustrating a flow of a relative position calculation process (including a coordinate-added X-ray image data calculation process by a magnetic field data processing device) by a magnetic field data processing system.

In step S701, a physician or the like inputs information (subject information) of the subject 200 to the X-ray image data processing device 120. The subject information input by a physician or the like includes a subject ID, a name, age, sex, height, weight, and the like.

In step S702, the physician or the like attaches marker coils 201 to the subject 200.

In step S703, a physician or the like performs X-ray imaging from the front of the subject 200 using the X-ray imaging unit 110.

In step S704, the X-ray imaging unit 110 generates X-ray image data 210 and transmits the generated X-ray image data to the X-ray image data processing device 120. As a result, the X-ray image data processing device 120 acquires the X-ray image data 210 (see reference numeral 901 in FIG. 9). The X-ray image data processing device 120 performs various image processes on the acquired X-ray image data 210 and transmits the processed X-ray image data to the magnetic field data processing device 140 (see the arrow 902 in FIG. 9).

In step S705, a physician or the like gets the subject 200 to lie flat on the back such that the vicinity of the spine of the subject 200 abuts on the position of the dewar 300. In addition, the physician or the like measures the magnetic field of the marker coils 201 attached to the subject 200 using the magnetic sensor array 130.

In step S706, the magnetic sensor array 130 generates magnetic field data 310 and transmits the generated magnetic field data 310 to the magnetic field data processing device 140 (see reference numerals 931 and 932 in FIG. 9).

In step S707, the magnetic field data processing device 140 executes a coordinate-added X-ray image data calculation process.

Specifically, in step S801 of FIG. 8, the coordinate-added X-ray image data calculator 141 acquires X-ray image data 210 from the X-ray image data processing device 120.

In step S802, the coordinate-added X-ray image data calculator 141 acquires the magnetic field data 310 from the magnetic sensor array 130.

In step S803, the coordinate-added X-ray image data calculator 141 generates magnetic field distribution data 910 based on the magnetic field data 310 (see reference numeral 941 in FIG. 9). In addition, the coordinate-added X-ray image data calculator 141 detects the position at which the intensity of the magnetic field peaks in the magnetic field distribution data 910. Note that the position at which the intensity of the magnetic field peaks in the magnetic field distribution data 910 corresponds to the positions of the marker coils 201 (see reference numeral 941 in FIG. 9).

With the point 330 as the origin, the coordinate-added X-ray image data calculator 141 calculates the distance to the position at which the intensity of the magnetic field peaks, and calculates the coordinates of the peak position. As a result, the coordinate-added X-ray image data calculator 141 calculates x and y coordinates of each of the marker coils 201. Note that the example of FIG. 9 illustrates that (xm1, ym1), (xm2, ym2), (xm3, ym3), and (xm4, ym4) are calculated as the x coordinate and the y coordinate of each of the marker coils 201 (see reference numeral 941 in FIG. 9).

In step S804, the coordinate-added X-ray image data calculator 141 detects each of the marker coils (reference numeral 211) reflected in the acquired X-ray image data 210.

In addition, the coordinate-added X-ray image data calculator 141 calculates the x coordinate and the y coordinate ((xm1, ym1) to (xm4, ym4)) of each of the calculated marker coils 201 at each of the positions of the marker coils (reference numeral 211) detected from the X-ray image data 210. In FIG. 9, an arrow 942 indicates that the calculated x and y coordinates of the marker coils 201 are reflected in the X-ray image data 210.

In step S805, the coordinate-added X-ray image data calculator 141 calculates coordinates of each pixel of the X-ray image data 210 (the x coordinate and the y coordinate of each of the pixels by setting the point 330 as the origin), based on the x coordinate and y coordinate reflected on the positions of the marker coils (reference numeral 211). As a result, the coordinate-added X-ray image data calculator 141 generates coordinate-added X-ray image data 920 (see reference numeral 941 in FIG. 9). That is, the coordinate-added X-ray image data 920 generated by the coordinate-added X-ray image data calculator 141 is relative position data adding a relative position (xy coordinates) to each of the pixels of the X-ray image data 210 with respect to the position of the point 330 of the magnetic sensor array 130 as the origin.

In FIG. 9, gridline indicating xy coordinates on the coordinate-added X-ray image data 920 are merely depicted for convenience of simplifying explanation, and hence, in the following description, such gridline are not indicated in the coordinate-added X-ray image data 920.

In step S806, the coordinate-added X-ray image data calculator 141 stores the generated coordinate-added X-ray image data 920 in the X-ray image data storage 144.

### 8. Illustration of Mesh Generation Process (Step S602)

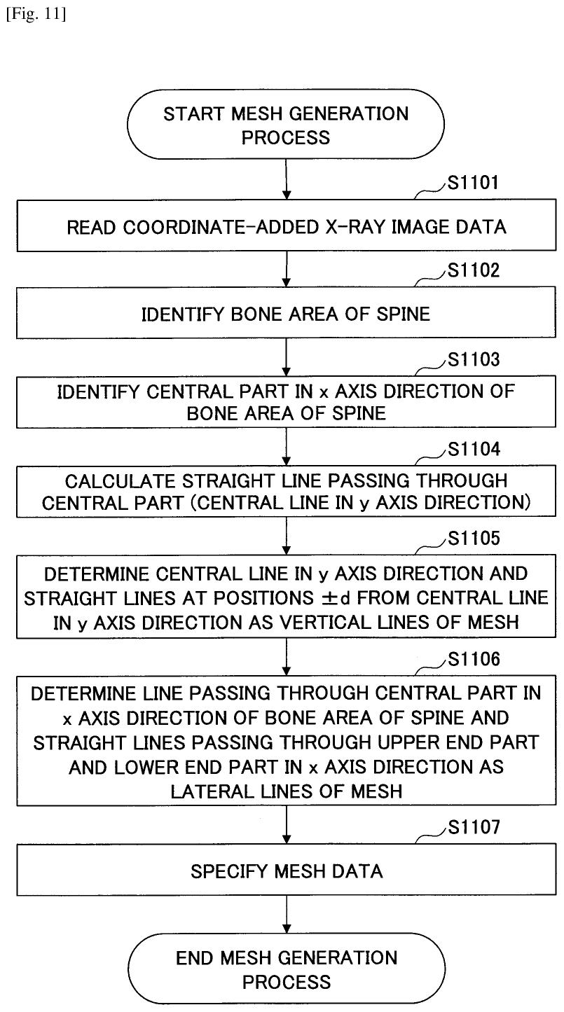

Next, details of the mesh generation process (step S602) will be described. FIG. 10 is a diagram illustrating a detailed functional configuration of a mesh generator configured to execute a mesh generation process. As illustrated in FIG. 10, the mesh generator 142 includes a coordinate-added X-ray image data reader 1001, a part identification unit 1002, and a mesh data specification unit 1003.

The coordinate-added X-ray image data reader 1001 reads coordinate-added X-ray image data 920 from the X-ray image data storage 144.

The part identification unit 1002 analyzes the read coordinate-added X-ray image data 920 to identify a predetermined part of the subject (a part that the physician or the like desires to observe for identifying the damaged part).

The mesh data specification unit 1003 generates a mesh based on gridlines passing through the identified predetermined part, thereby generating a mesh defining grid points for the predetermined part. In addition, the mesh data specification unit 1003 specifies positions of the grid points of the generated mesh based on the coordinate-added X-ray image data 920, thereby specifying mesh data.

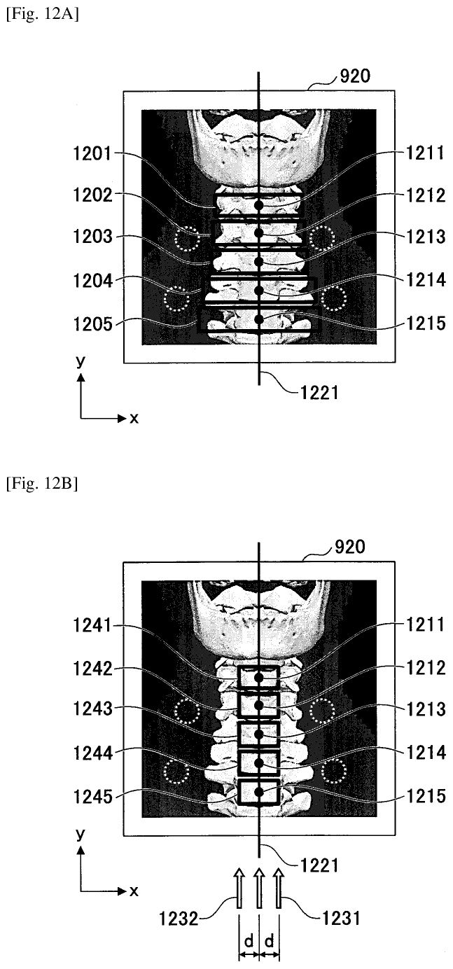

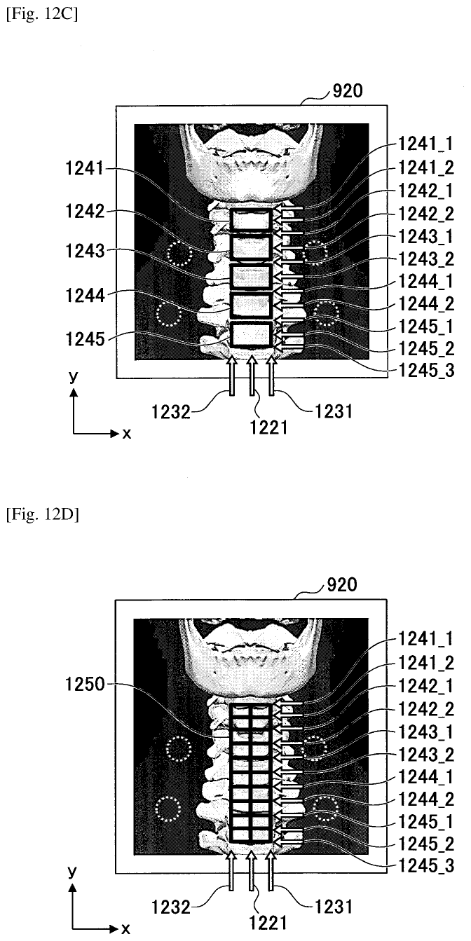

Hereinafter, the details of the functions of respective units (the coordinate-added X-ray image data reader 1001, the part identification unit 1002, and the mesh data specification unit 1003) of the mesh generator 142 will be described with reference to FIGS. 11 and 12.

FIG. 11 is a flowchart illustrating a flow of a mesh generation process by respective units of a mesh generator. FIGS. 12A to 12D are diagrams schematically illustrating a flow of a mesh generation process by respective units of a mesh generator.

In step S1101, the coordinate-added X-ray image data reader 1001 reads coordinate-added X-ray image data 920 from the X-ray image data storage 144.

In step S1102, the part identification unit 1002 analyzes the coordinate-added X-ray image data 920 to identify vertebrae areas. Note that in the present embodiment, it is assumed that the part identification unit 1002 identifies vertebrae areas using a known identification method. In FIG. 12A, areas 1201 to 1205 indicate the vertebrae areas identified by the part identification unit 1002.

In step S1103, the part identification unit 1002 identifies a central part in the x axis direction of the identified vertebrae areas 1201 to 1205. Points 1211 to 1215 in FIG. 12A indicate the central area identified by the part identification unit 1002 for each of the vertebrae areas 1201 to 1205.

In step S1104, the mesh data specification unit 1003 calculates a gridline (the center line in y-axis direction) passing through the identified central part. In FIG. 12A, the gridline 1221 indicates a center line in an y-axis direction passing through the points 1211 to 1215.

In step S1105, the part identification unit 1002 identifies a part at a +d position from the gridline 1221 in the x axis direction and a part at a −d position from the gridline 1221 in the x axis direction. In FIG. 12B, an arrow 1231 indicates a +d position from the gridline 1221 in the x axis direction. Similarly, an arrow 1232 indicates a −d position from the gridline 1221 in the x axis direction.

The mesh data specification unit 1003 calculates gridlines substantially parallel to the gridline 1221 and extending from the position of the arrow 1231 and the position of the arrow 1232 in the y axis direction (gridlines passing through a part at a +d or −d position in the x axis direction with respect to the gridline 1221). These gridlines that are calculated by the mesh data specification unit 1003 as vertical lines for generating a mesh, together with the center line in the y axis direction. The mesh data specification unit 1003 also generates rectangular areas 1241 to 1245 with the arrows 1231 and 1232 set as two end positions based on these gridlines and on the vertebrae areas 1201 to 1205.

In step S1106, the part identification unit 1002 identifies upper and lower end parts and the central part in the y axis direction for each of the rectangular areas 1241 to 1245. In FIG. 12C, the arrows 1241_1, 1241_2, and 1242_1 indicate the position of the upper end part, the position of the central part, and the position of the lower end part, respectively, of the rectangular area 1241 in the y axis direction. Further, the arrows 1242_1, 1242_2, and 1243_1 indicate the position of the upper end part, the position of the central part, and the position of the lower end part, respectively, of the rectangular area 1242 in the y axis direction. Likewise in the following, the arrows 1243_1 to 1245_3 indicate the positions of the upper end part, the positions of the central part, and the positions of the lower end part, respectively, of the rectangular areas 1243 to 1245 in the y axis direction.

The mesh data specification unit 1003 calculates gridlines that are substantially orthogonal to the gridline 1221 and extend from the position of the arrow 1241_1 to the position of 1245_3 in the x axis direction (gridlines passing through the upper and lower end parts, and the central parts of the rectangular areas 1241 to 1245 along the y axis direction). These gridlines calculated by the mesh data specification unit 1003 are horizontal lines for generating a mesh.

In step S1107, the mesh data specification unit 1003 generates a mesh 1250 (see FIG. 12D) based on the vertical lines and the horizontal lines for generating the mesh, and determines the position of each of the grid points. Note that the grid point is an intersection point of the vertical line and the horizontal line for generating the mesh 1250 and is a calculated position at which the current value is calculated for generating the reconfigured data based on the magnetic field data 320.

As described above, the part identification unit 1002 identifies a predetermined part (a part that a physician or the like desires to observe for specifying a damaged part), and the mesh data specification unit 1003 generates a mesh based on gridlines passing through the predetermined part. As a result, the mesh data specification unit 1003 may generate a mesh having a grid point set as the position of each of the parts identified by the part identification unit 1002. That is, the mesh data specification unit 1003 may set a part (a position suitable for specifying a damaged part) that a physician or the like desires to observe to specify a damaged part as a calculated position for generating reconfigured data.

In addition, the mesh data specification unit 1003 specifies coordinates indicating positions of the grid points of the generated mesh 1250 (see FIG. 12D) based on the coordinate-added X-ray image data 920, thereby specifying mesh data. The mesh data may be represented by a set of coordinates (x coordinates and y coordinates with the point 330 as the origin) indicating the positions of grid points of the mesh 1250, for example.

The mesh data specification unit 1003 stores the specified mesh data in the mesh data storage 145.

The mesh generator 142 specifies the mesh data based on the mesh generation process as described above according to the following reasons.

To specify a damaged part in the spine of a subject based on the reconfigured data, the physician or the like determines any one of a part inside a vertebra of the subject 200, an intervertebral part, and a part inside or outside the vertebra that stagnates neural transmission. Accordingly, for generating reconfigured data, it is desirable that the current value is calculated at the central part of the vertebra, the intervertebral part, and the parts at the opposite ends of the vertebra where the nerves enter the spine.

Therefore, the mesh generator 142 identifies the central part of the vertebra, calculates gridlines (the gridline 1221, and gridlines at positions indicated by arrows 1241_2, 1242_2, . . . 1245_2) passing through the part, and generates a mesh 1250. Further, the mesh generator 142 identifies the intervertebral parts and parts at the opposite ends of the vertebra, and calculates gridlines passing through these parts (gridlines at positions indicated by arrows 1221, 1231, 1232, 1241_1, 1242_1, . . . 1245_3) to generate a mesh 1250.

As described above, the mesh generator 142 calculates a current value based on the magnetic field data 320 with the positions of grid points of the mesh 1250 as calculated positions. Accordingly, based on the reconfigured data generated using the mesh 1250 as described above, the physician or the like may check the presence or absence of the electric current sources at each grid point. As a result, the physician or the like may be able to ascertain any one of a part inside the subject's vertebra, an intervertebral part, or a part inside or outside the vertebra that stagnates the neural transmission so as to specify a damaged part.

For example, in a case where a physician or the like focuses on a predetermined vertebra, it is assumed that the physician or the like is able to identify electric current sources at an intervertebral part below the predetermined vertebra, but the physician or the like is not able to identify the electric current sources at an intervertebral part above the predetermined vertebra. In this case, the physician or the like may be able to ascertain the neural transmission stagnating in the vertebra, thereby specifying the vertebra as a damaged part.

Further, by appropriately setting the value of the distance d specifying the positions of the arrow 1231 and the arrow 1232, the physician or the like may be able to ascertain whether the neural transmission stagnates at the part where the nerves enter the vertebrae. In this case, the value of the distance from the central part of the vertebra to the part where the nerves enter is set as the distance d, for example. Since there is not a significant difference between individuals in the distance from the central part of the vertebra to the part where the nerves enter, the distance d may be a fixed value; however, the distance d may be calculated based on a predetermined ratio with respect to the width of the vertebra.

### 9. Illustration of Reconfiguration Process (Step S603)

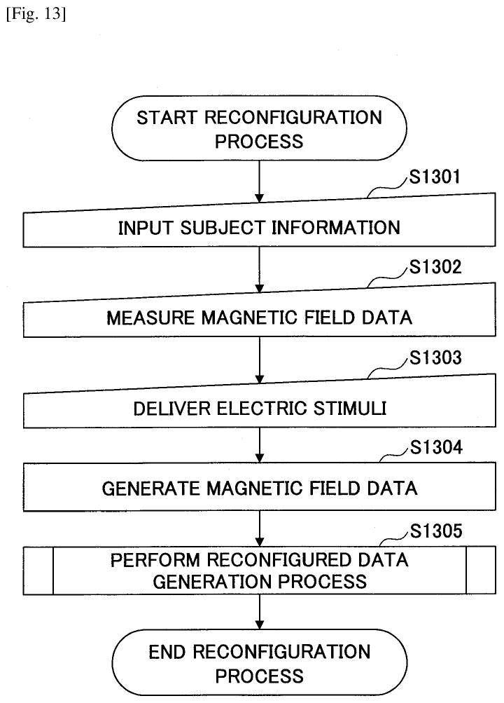

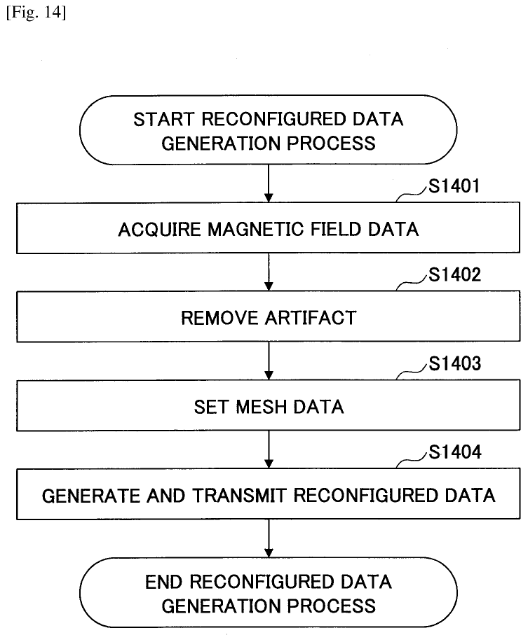

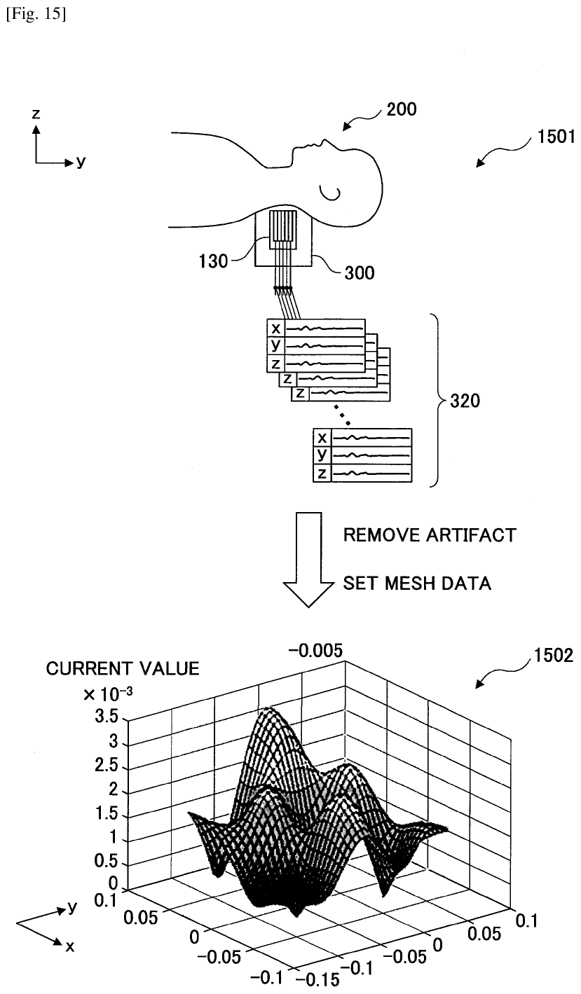

Next, a reconfiguration process (step S603) will be described in detail using FIGS. 13 and 14, with reference to FIG. 15. FIG. 13 is a flowchart illustrating a flow of a reconfiguration process by a magnetic field data processing system. FIG. 14 is a flowchart illustrating a flow of a reconfigured data generation process by a magnetic field data processing device. FIG. 15 is a diagram schematically illustrating a flow of a reconfiguration process (including a reconfigured data generation process by a magnetic field data processing device) by a magnetic field data processing system.

In step S1301, a physician or the like inputs information (subject information) of the subject 200 to the magnetic field data processing device 140.

In step S1302, the physician or the like removes the marker coils 201 from the subject 200 lying flat on the back such that the vicinity of the spine of the subject 200 abuts on the position of the dewar 300. In addition, the physician or the like starts measuring the magnetic field data using the magnetic sensor array 130 (see reference numeral 1501 in FIG. 15).

In step S1303, the physician or the like attaches an electrode to a predetermined stimulation part of the subject 200 (e.g., the left arm of the subject 200) and applies an electrical stimulus to the subject 200.

In step S1304, the magnetic sensor array 130 generates magnetic field data 320 and transmits the generated magnetic field data 320 to the magnetic field data processing device 140 (see reference numeral 1501 in FIG. 15).

In step S1305, the reconfigured data generator 143 of the magnetic field data processing device 140 executes a reconfigured data generation process.

Specifically, in step S1401 of FIG. 14, the reconfigured data generator 143 acquires the magnetic field data 320.

In step S1402, the reconfigured data generator 143 removes artifacts included in the magnetic field data 320.

In step S1403, the reconfigured data generator 143 reads the mesh data stored in the mesh data storage 145.

In step S1404, the reconfigured data generator 143 reconfigures electric current sources from the magnetic field data 320 using the read mesh data, thereby calculating a current value at each of grid points to generate reconfigured data. The reconfigured data 1502 depicted in FIG. 15 is an example in which reconfigured data are generated using a mesh, which is generated by expanding vertical lines and horizontal lines defining the mesh 1250 further in the x axis direction and the y-axis direction, respectively. In the reconfigured data 1502, current values are calculated as calculated positions with respect to a position of the part that the physician or the like desires to observe. Accordingly, by referring to the reconfigured data 1502, the physician or the like may be able to specify a damaged part of the subject 200 (e.g., to locate a part of the spine of the subject 200 where neural transmission fails).

The reconfigured data generator 143 transmits the generated reconfigured data 1502 to the server apparatus 150 in association with the subject information.

### 10. Outline

As is apparent from the above description, the magnetic field data processing system 100 according to the present embodiment
includes an X-ray imaging unit configured to perform X-ray imaging on a subject with marker coils attached thereto to generate X-ray image data including X-ray image data of a predetermined part (a part that a physician or the like desires to observe for specifying a damaged part) of the spine of the subject.
calculates, based on the generated X-ray image data and the magnetic field distribution data of the marker coils, relative position data (coordinate-added X-ray image data) indicating a relative position of the subject with respect to the magnetic sensor array (x coordinate, y coordinate) for measuring the subject using the magnetic sensor array.
identifies a predetermined part of the spine of the subject identified in the coordinate-added X-ray image data, and specifies a relative position (x coordinate, y coordinate) of the identified predetermined part with respect to the magnetic sensor array based on the coordinate-added X-ray image data.
generates a mesh with the identified relative position as a grid point (calculated position), reconfigures the electric current sources from the magnetic field data measured by the magnetic sensor array using the generated mesh, and generates reconfigured data.

As described above, in the magnetic field data processing system 100 according to the present embodiment, a mesh is generated based on the relative position of the predetermined part of the subject with respect to the magnetic sensor array. Accordingly, in the magnetic field data processing system 100 of the present embodiment, a part (of the subject) that a physician or the like desires to observe for specifying a damaged part may be set as a calculated position for generating reconfigured data. As a result, according to the magnetic field data processing system 100 of the present embodiment, it is possible to generate the reconfigured data in which the electric current sources are reconfigured at the calculated position suitable for specifying the damaged part of the subject.

### Other Embodiments

In the above-described embodiment, the X-ray imaging unit 110 is disposed in the magnetic field data processing system 100, such that the magnetic field data processing system 100 including the X-ray imaging unit 110 may be able to generate image data including a predetermined part of the spine of the subject. However, the configuration for generating the image data including a predetermined part of the spine of the subject 200 is not limited to the X-ray imaging unit 110, and other measurement devices capable of visualizing a predetermined part of the spine of the subject 200 may be disposed in the magnetic field data processing system 100, in place of the X-ray imaging unit 110.

Further, in the above embodiment, a predetermined part of the spine of the subject 200 is identified in the coordinate-added X-ray image data so as to generate a mesh, which includes the position of the identified predetermined part defined as a grid point (calculated position). However, the mesh generation method is not limited to this example. For example, in a case where the identified predetermined part is included in the grid points, further refined grid points may be set to generate the mesh.

Further, in the above embodiment, a predetermined part of the spine of the subject 200 is identified in the coordinate-added X-ray image data, a mesh is generated based on the identified predetermined part, and then grid points (calculated positions) are defined (as the position of the identified predetermined part). Specifically, in the present embodiment, gridlines passing through the identified predetermined part are calculated, a mesh is generated based on the calculated gridlines, the positions of the respective grid points are determined, and the positions of the determined grid points are determined as the calculated positions for generating reconfigured data. However, without generating a mesh, the position of the identified predetermined part may be directly used as the calculated position for calculating the reconfigured data.

In the configuration according to the above embodiment, the part identification unit 1002 identifies a predetermined part of the spine of the subject 200 in the coordinate-added X-ray image data. However, the method of identifying a predetermined part of the spine of the subject 200 is not limited to this example. For example, a physician or the like designates the position of a predetermined part of the spine of the subject while referring to the coordinate-added X-ray image data, and the part identification unit 1002 identifies the position of the part designated by the physician or the like.

In the above embodiment, a case where the magnetic sensor array 130 is used as a biological sensor has been described. However, the present embodiment may be applied to a case where the electric current sources are reconfigured using biological data measured using another biological sensor (e.g., electroencephalograph).

It should be noted that the present invention is not limited to the configurations described in the above embodiments, such as combinations with other elements, and the like. With respect to these points, alterations or modifications may be made within a scope of the claims in accordance with appropriately determined forms of application without departing from the gist of the present invention.

### REFERENCE SIGNS LIST

100 magnetic field data processing system

110 X-ray imaging unit

120 X-ray image data processing device

130 magnetic sensor array

140 magnetic field data processing device

141 coordinate-added X-ray image data calculator

142 mesh generator

143 reconfigured data generator

150 server apparatus

200 subject

210 X-ray image data

310 magnetic field data

320 magnetic field data

330 origin

910 magnetic field distribution data

920 coordinate-added X-ray image data

1001 coordinate-added X-ray image data reader

1002 part identification unit

1003 mesh data specification unit

1502 reconfigured data

The present application is based on and claims the benefit of priority of Japanese Priority Application No. 2016-235112 filed on Dec. 2, 2016, the entire contents of which are hereby incorporated herein by reference.

1. A biological data processing device comprising:
a calculator configured to calculate relative position data indicating a relative position of a subject with respect to a biological sensor for measuring the subject using the biological sensor;
a specifying unit configured to generate a mesh composed of grid lines based on lines passing through a predetermined part of the subject to specify a relative position of the predetermined part of the subject and relative positions of grid points of the mesh with respect to the biological sensor, based on the relative position data; and
a generator configured to estimate electric current sources from biological data measured by the biological sensor to generate electric current data at the specified relative positions.

2. (canceled)

3. The biological data processing device according to claim 1,
wherein the biological sensor is a magnetic sensor, and
wherein the specifying unit
acquires X-ray imaged data generated by performing X-ray imaging on the subject with position detection markers being attached to the subject, and magnetic field distribution data generated based on magnetic field data measured by the magnetic sensor with the position detection markers being attached to the subject, and
calculates the relative position data based on positions of the position detection markers in the X-ray image data and positions of the position detection markers in the magnetic field distribution data.

4. The biological data processing device according to claim 3,
wherein the specifying unit specifies a relative position of a predetermined part of the subject with respect to the biological sensor based on the X-ray image data.

5. The biological data processing device according to claim 1,
wherein the predetermined part includes respective central parts of the vertebrae of the subject.

6. The biological data processing device according to claim 1,
wherein the predetermined part includes an intervertebral part of the subject.

7. The biological data processing device according to claim 5, wherein
the predetermined part includes a part at a predetermined distance from a center line connecting the central parts of the vertebrae of the subject.

8. A biological data processing system comprising:
a calculator configured to calculate relative position data indicating a relative position of a subject with respect to a biological sensor for measuring the subject using the biological sensor;
a specifying unit configured to generate a mesh composed of grid lines based on lines passing through a predetermined part of the subject to specify a relative position of the predetermined part of the subject and relative positions of grid points of the mesh with respect to the biological sensor, based on the relative position data; and
a generator configured to estimate electric current sources from biological data measured by the biological sensor to generate electric current data at the specified relative positions.

9. A non-transitory storage medium storing a biological data processing program, which when processed by processors causes a computer to execute a process comprising:
calculating relative position data indicating a relative position of a subject with respect to a biological sensor for measuring the subject using the biological sensor;
generating a mesh composed of grid lines based on lines passing through a predetermined part of the subject to specify a relative position of the predetermined part of the subject and relative positions of grid points of the mesh with respect to the biological sensor, based on the relative position data; and
estimating electric current sources from biological data measured by the biological sensor to generate electric current data at the specified relative positions.
